(19) United States
(12) Patent Application Publication
(10) Pub. No.: US 2011/0009773 A1
(43) Pub. Date: Jan. 13, 2011

(54) IMPLANTABLE SENSING MODULES AND METHODS OF USING

(75) Inventors: Robert William Hower, Brighton, MI (US); Navid Yazdi, Ann Arbor, MI (US); Yafan Zhang, Troy, MI (US)

Correspondence Address: HARTMAN & HARTMAN, P.C., 552 EAST 700 NORTH, VALPARAISO, IN, 46383, US

(73) Assignee: EVIGIA SYSTEMS, INC., Ann Arbor, MI (US)

(21) Appl. No.: 12/856,011

(22) Filed: Aug. 13, 2010

Related U.S. Application Data
(63) Continuation-in-part of application No. 11/671,130, filed on Feb. 5, 2007.
(60) Provisional application No. 61/272,066, filed on Aug. 13, 2009.
(60) Provisional application No. 60/765,244, filed on Feb. 4, 2006.

(51) Int. Cl. A61B 5/00

(52) U.S. Cl. 600/578

(57) ABSTRACT

Implantable sensing modules and methods for monitoring various physical parameters, including physical parameters of a living body and environmental parameters to which the living body may be subjected, for example, impacts. A method for monitoring impacts to which a living body is subjected entails the use of an implantable sensing module that has a rigid housing containing at least one energy storage device and at least one electromechanical sensing element that is responsive to impacts. The module generates data corresponding to impacts to which the electromechanical sensing element is subjected, and records the data in memory. The module is preferably implanted in a living body so that the module is connected to a rigid portion of the living body, in particular, a bone or tooth.

### CROSS REFERENCE TO RELATED APPLICATIONS

This application claims the benefit of U.S. Provisional Application No. 61/272,066, filed Aug. 13, 2009, and is a continuation-in-part patent application of co-pending U.S. patent application Ser. No. 11/671,130, filed Feb. 5, 2007, which claimed the benefit of U.S. Provisional Application No. 60/765,244, filed Feb. 4, 2006. The contents of these prior applications are incorporated herein by reference.

### BACKGROUND OF THE INVENTION

The present invention generally relates to electromechanical devices, such as micro-electromechanical systems (MEMS) and nano-electromechanical systems (NEMS). More particularly, this invention relates to implantable sensing modules capable of being implanted for the purpose of monitoring physical parameters of a living body and/or monitoring environmental parameters to which the body may be subjected, a particular but nonlimiting example of which is impacts sustained by the body.

Wireless sensor systems are known that have the capability for high reliability, efficiency, and performance. Such systems can be employed in a wide range of applications including supply-chain and logistics, industrial and structural monitoring, healthcare, homeland security, and defense. Generally, it is desired to minimize the power dissipation, size, and cost of these systems by making them low-power and/or operate without a battery. Furthermore, in many applications a batteryless operation is needed due to lack of battery replacement feasibility, or to meet stringent cost, form factor, and lifetime requirements. One approach to address this need is scavenging energy from environmental sources such as ambient heat, radio and magnetic waves, vibrations, and light. However, in many situations, these environmental energy sources are not adequately available to power a sensor. Another approach is to remotely power wireless sensor systems by inductive or electromagnetic coupling, storing energy on a suitable energy storage device, such as one or more integrated capacitors or miniature batteries, and performing sensor operations over short periods of time prior to minimize that discharge rate of the energy storage device.

Because of the size and complexity of many implantable sensing systems and the need for battery replacement to power the systems, individuals and the medical community have been reluctant to implant sensing systems into the human body. In addition, a living body will reject and encapsulate an implanted system in a matter of days, often interfering with their operation and, in the case of chemical sensors, rendering them impractical. However, there are many types of sensors that can monitor the body that do not require direct access to bodily fluids. For example, micro-electromechanical system (MEMS) and nano-electromechanical system (NEMS) sensors have been developed for incorporation into the body for continuous monitoring. These sensors include, but are not limited to temperature, acceleration (including impact or shock), vibration, impact, motion, and blood/capsule pressure sensing.

There are many health problems that could benefit from real-time temperature monitoring, including determining overheating/heat stroke and/or hypothermia in athletes and other individuals. The simplest form of temperature monitor is placed directly on the skin, though a drawback of this method is that the sensor will not provide an accurate indication of body temperature because skin temperature is influenced by environmental conditions such as weather conditions. To accurately indicate core body temperature, a temperature sensing device may be swallowed. However, such a device must be disposable for acceptance and therefore its cost must be very low. Furthermore, the sensed temperature can fluctuate depending on where the sensing device is in the digestive tract, and the sensor package must endure very harsh conditions of the digestive tract (highly acidic and highly basic). Finally, there is the possibility of injury to the individual in the event the sensor package should break.

Acceleration (including impact or shock) is another important parameter of interest in the healthcare industry. For example, impact monitoring can be used to indicate if an individual has suffered from head trauma, a child has been shaken, or an elderly person has fallen. However, existing impact sensing systems are not typically implanted because they were large, require major surgery, and can incur significant health risk to the individual. Existing systems also require batteries that must be changed on a fairly regular basis. In most situations, patients will not want to submit themselves to the risks of surgery if the system is only capable of operating for a few days. Consequently, currently available systems are typically limited to monitoring acceleration or impact on equipment worn by an individual, such as a helmet of the type used in hockey or American football. These systems are typically heavy, consume a significant amount of power, and are very expensive. Furthermore, the transfer function from motion of the helmet to motion of the head is different for every individual, and can depend on the fit of the helmet, tightness of the chin strap, how the helmet is worn, and many other factors varying from individual to individual.

### BRIEF SUMMARY OF THE INVENTION

The present invention provides implantable sensing modules and methods for monitoring various physical parameters, including physical parameters of a living body and environmental parameters to which the living body may be subjected, for example, impacts.

According to a first aspect of the invention, a method is provided for monitoring impacts to which a living body is subjected. The method entails the use of an implantable sensing module that comprises a rigid housing containing at least one energy storage device, at least one electromechanical sensing element that is responsive to impacts, means for generating outputs corresponding to impacts to which the electromechanical sensing element is subjected, and means for recording the outputs. The module is implanted in a living body so that the module is located internally within the living body and is connected to a rigid portion of the living body, in particular, a bone or tooth. Impacts to the living body are then monitored by monitoring levels of impacts to which the electromechanical sensing element is subjected within the living body. Outputs corresponding to the levels of the impacts sensed by the electromechanical sensing element are then produced, and the outputs stored in the recording means within the module. These outputs can then be wirelessly retrieved from the recording means while the module remains implanted in the living body.

In view of the above, it can be seen that an implantable sensing module according to the first aspect of the invention is capable of very accurately monitoring impacts to a body as a result of being directly attached to a rigid surface of the body of head impacts, thereby improving diagnosis and treatment methodologies. In a preferred but optional embodiment, the module is also configured to operate with minimal power so that power is available for system operation over longer periods of time. In a particularly preferred embodiment, the electromechanical sensing elements scavenge power from the body, providing a continuous monitoring capability over extended periods of time. The module is preferably configured to quickly and accurately record data, yet can also be small enough to be implanted using a needle or through a small incision.

According to a second aspect of the invention, a method is provided for monitoring at least one external input to a living body, in particular, a physical parameter of the body or an environmental parameter to which the body is subjected. The method entails the use of an electromechanical system module that comprises at least one integrated energy storage device and a plurality of integrated electromechanical switches. The electromechanical switches define open electrical paths and are operable to define closed electrical paths to produce outputs in response to the external input. Furthermore, the electromechanical switches have different levels of sensitivity to the external input. After implanting the module in a living body, the body may be subjected to the external input that causes two or more of the electromechanical switches to define at least two of the closed electrical paths in response to different input levels of the external input. The closed electrical paths produce at least two outputs corresponding to the different input levels of the external input. Finally, the outputs are obtained from the module.

In view of the above, it can be seen that an implantable sensing module according to the second aspect of the invention is well suited for implantation in a living body as a result of its size being minimized and its operation extended as a result of the electromechanical switches operating only during sensing events. As such, the implantable sensing module is capable of longer periods of operation compared to conventional implantable sensors that require continuous power from a battery, and is capable of a far greater level of functionality as compared to implantable sensors that do not have any internal energy storage capability. As with the module according to the first aspect of the invention, the module can be configured to quickly and accurately record data, yet can also be small enough to be implanted using a needle or through a small incision.

According to another aspect of the invention, an implantable sensing module is provided for monitoring impacts to which a living body is subjected. The module includes a housing and at least one energy storage device and at least one set of electromechanical sensing elements within the housing. The sensing elements are responsive to impacts, each defines an open electrical path when not subjected to an impact, and each is operable to define a closed electrical path that produces an output in response to an impact only while the sensing element is subject to the impact and if the impact exceeds a threshold of the sensing element. Each sensing element again defines the open electrical path thereof so as not to produce an output when no longer subject to the impact that exceeded its threshold. The housing further contains means for generating data corresponding to the outputs of the sensing elements, and means for recording the data. The sensing elements, generating means, and recording means are powered only by the energy storage device when, respectively, producing the output, generating the data, and recording the data in response to an impact that exceeded the threshold of one or more of the sensing elements.

Other objects and advantages of this invention will be better appreciated from the following detailed description.

### BRIEF DESCRIPTION OF THE DRAWINGS

FIGS. 1 and 2 represent block diagrams of implantable sensing modules in accordance with an embodiment of the invention.

FIGS. 3 and 4 schematically represent perspective and side views, respectively, of an electromechanical switch configured as a temperature sensing element that is suitable for use in the modules of FIGS. 1 and 2.

FIGS. 5 and 6 schematically represent the switch of FIGS. 3 and 4 at opposite extremes of its operating range in response to two threshold temperature conditions.

FIGS. 7 and 8 schematically represent perspective and side views, respectively, of an electromechanical switch configured as an impact sensing element that is suitable for use in the modules of FIGS. 1 and 2.

FIGS. 9 and 10 schematically represent the switch of FIGS. 7 and 8 at opposite extremes of its operating range in response to two threshold impact and/or acceleration conditions.

FIGS. 11 and 12 schematically represent perspective and side views, respectively, of an electromechanical switch configured as a chemical sensing element that is suitable for use in the modules of FIGS. 1 and 2.

FIGS. 13 and 14 schematically represent the switch of FIGS. 11 and 12 at opposite extremes of its operating range in response to two threshold conditions.

FIG. 15 schematically represents a side view of an electromechanical switch configured as a pressure sensing element that is suitable for use in the modules of FIGS. 1 and 2.

FIG. 16 schematically represents the MEMS switch of FIG. 15 at one extreme of its operating range in response to a threshold pressure condition.

FIGS. 17 and 18 schematically represent side views of an alternative electromechanical switch that is suitable for use in the modules of FIGS. 1 and 2.

FIGS. 19 and 20 schematically represent side views of switches according to FIGS. 3 through 18, further equipped with means for tuning their sensing threshold using a stored charge according to an optional aspect of the invention.

FIG. 21 schematically represents a plan view of MEMS switches for use in a digital sensor array of the module of FIG. 2, and configured for sensing linear and angular acceleration with six degrees of freedom.

### DETAILED DESCRIPTION OF THE INVENTION

The present invention provides miniature implantable sensing modules whose small size enables the modules to be placed into a living body, preferably to a bone, tooth or other rigid surface where it can monitor and generate data relating to an external input, such as physiological parameters of the body and/or environmental parameters to which the body may be subjected. The modules make use of an energy storage device and one or more electromechanical sensing elements. The modules also preferably make use of non-volatile memory to store the data and a wireless communication system that enables the data to be retrieved from the modules by an external reader. The components of the modules are preferably selected so that the modules require very little power for their operation, enabling the modules to remain implanted and operable for long periods of time, potentially on the order of years, without need for replacement.

As will be evident from the following description, a particular object of this invention is to extend the life of an implantable sensing module that employs an energy storage device, for example, a capacitor, battery or other suitable energy storage device. As will be discussed in more detail below, one such approach is to configure the electromechanical sensing elements to operate in response to the external input without drawing power from the energy storage device. Preferred electromechanical sensing elements are micro-electromechanical system (MEMS) and nano-electromechanical system (NEMS) sensing elements. As used herein, the terms MEMS and NEMS denote miniature devices generally on a scale of less than a millimeter and less than a micrometer, respectively, that incorporate both electronic and mechanical functionalities, and are produced by micromachining techniques, such as bulk etching and surface thin-film etching.

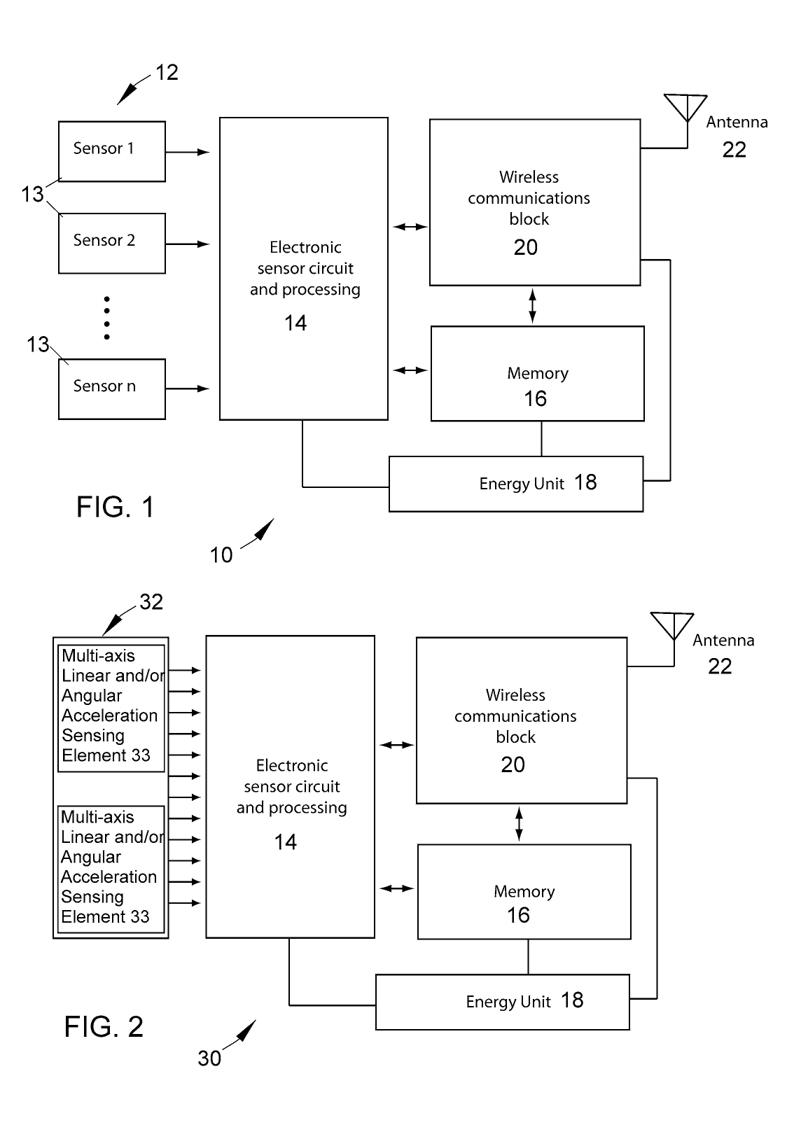

FIG. 1 represents a block diagram of an implantable sensing module 10 according to one embodiment of the invention. The system architecture of the module 10 includes an array 12 of digital sensing elements 13 connected to a controller 14 that electronically monitors and processes the outputs generated by the elements 13, and then stores data corresponding to the outputs into memory 16. The sensing elements 13 can be fabricated on a single circuit chip or multiple circuit chips and mounted within a rigid housing (not shown) in which the components of the module 10 are packaged. Individual chips on which the sensing elements 13 are fabricated can be micropackaged during their batch-manufacturing prior to mounting within the module housing. The controller 14 can also be conventionally fabricated on an integrated circuit chip. The memory 16 preferably comprises nonvolatile digital memory devices, such as one or more CMOS chips, though other types of memory devices can also be used. Data processing performed by the controller 14 may include eliminating any false outputs and filtering the data before storage to reduce the memory required to store the data in the memory 16. The controller 14 preferably utilizes integrated ultra-low power digital signal processing to compress and store the data. The module 10 also comprises an energy management unit 18 that contains an energy storage device (not shown) for supplying DC power to the controller 14, as well as to a wireless communications block 20 adapted to transmit the data through an antenna 22 to an external reader unit (not shown). The energy storage device may be a capacitor, battery or any other suitable type of power storage device. Along with the one or more sensing element chips, the antenna 22 and the chips on which the controller 14, memory 16 and energy management unit 18 are fabricated can all be packaged within the same housing.

Wireless communication between the module 10 and the reader unit may be through a passive RFID communications protocol, though other wireless protocols are also foreseeable. RFID standard (ISO-15693) supports simultaneous data collection by a single reader unit from up to fifteen modules 10 having unique electronic ID codes. When a communications (e.g., interrogation) signal generated by a reader unit is received by the wireless communications block 20, the data stored in the memory 16 is accessed. The wireless communications block 20 can also be used to scavenge energy from the communications signal received from the reader unit and store the energy into the energy storage device (e.g., capacitor, battery, etc.) within the energy storage unit 18. The module 10 may also be electronically configurable through its wireless link to initialize the sensing elements 13 and their sensing ranges, designate the parameters that are to be recorded in the memory 16, and reset the sensing elements 13 and memory 16 as may be desired, for example, after data have been uploaded to the reader unit.

FIG. 2 represents a block diagram of an implantable sensing module 30 according to another embodiment of the invention. For convenience, identical reference numerals are used in FIG. 2 to denote the same or functionally equivalent elements described for the module 10 of FIG. 1. The module 30 of FIG. 2 differs from the embodiment of FIG. 1 by identifying the array of sensing elements as an array 32 of digital acceleration sensing elements 33, which in combination preferably provide a six-degree of freedom sensing capability. As will be discussed in reference to FIG. 21, in one embodiment of the invention the module 30 has an integrated six-axis acceleration capability provided with sensing elements 33 comprised of a combination of three linear and three angular acceleration sensors. As with the sensing elements 12 of FIG. 1, the sensing elements 33 can be fabricated on a single circuit chip or multiple circuit chips.

The overall the combination of small-size, light-weight, wireless data and command link, and electronic configurability enable the modules 10 and 30 to be useful for implantation into a living body, as well as embedding in various personnel and protective gear, sections of vehicles/equipment, etc. As noted above, in preferred embodiments of the modules 10 and 30, the memory 16 is able to store the data generated by the sensing elements 12/32 even if there is no external power supplied to the module 10 for extended periods of time. In this manner, the modules 10 and 30 are particularly well suited for implantation in the human body. In particular, the implantable wireless sensing modules of the type described in reference to FIGS. 1 and 2 can be used for many purposes, including tracking and recording one or more of a variety of physiological and environmental parameters. Nonlimiting examples include pressure for determining blood pressure, acceleration (including impacts and vibration), and temperature. Preferred acceleration sensing elements 33 have sensing capabilities of a range of about 0.1 g to 1000 g over durations ranging from about 1 μs to several or more seconds. For use within a living body, temperature measurements with accuracies of under 0.01° C. are preferred over a range of about 22° C. to about 52° C. is desirable.

Various potential locations are possible for the implantation of the modules 10 and 30. For accurately monitoring head trauma, placement of the modules 10 and 30 is preferably by direct attachment to the skull to improve the correlation between the impact sensed by the sensing elements 13 and 33 and the actual impact to which the brain is subjected. By placing the modules 10 and 30 containing highly sensitive sensing elements 13 and 33 directly to the skull to monitor its movement, a more accurate picture of potential brain injury can be determined. The invention also encompasses the attachment of the modules 10 and 30 to other bones to more accurately monitor impacts to which they are subjected, as well as monitor activity to determine or assess a wide range of maladies. The modules 10 and 30 can be fabricated to be sufficiently small to permit implantation with a needle or a small incision.

Implantation or placement of the modules 10 and 30 on a tooth is also within the scope of the invention. Because teeth are rigidly attached to the skull, attachment of the modules 10 and 30 to a tooth can provide excellent correlation between movement, acceleration and impact sensed by the sensing elements 13 and 33 and impacts to which the brain is subjected to assess the risk of brain injury.

In each case, because the modules 10 and 30 are implanted beneath the skin, there is excellent compliance for monitoring patients, as well as excellent correlation to external inputs often of interest, for example, internal temperature, pressure, and bone movement.

The functionality and life of the modules 10 and 30 can be considerably enhanced by configuring the sensing elements 13 and 33 to be scalable. According to preferred aspects of the invention, scalability, functionality and power efficiency of the sensing elements 13 and 33 can be greatly enhanced by configuring the sensing elements as switches with direct digital outputs covering a wide dynamic range. By configuring the sensing elements 13 and 33 to directly respond to external inputs without the requirement for power to be supplied to the elements 13 and 33, an ultra-low power electromechanical sensing system is provided that is entirely event-driven. As such, preferred sensing elements 13 and 33 extract energy from the external input they are intended to sense (e.g., pressure pulses, acceleration (impact, shock, vibration, movement, etc.), temperature, chemical species, blood alcohol level, etc.) to provide a direct digital output. When used to sense movement and acceleration (including impact or shock), switches are also capable of providing the advantage of minimum latency and capturing the rising edge of a shock/impact impulse.

U.S. Pat. Nos. 7,495,368 and 7,619,346 and U.S. patent application Ser. No. 11/671,130 disclose electromechanical switches particularly suitable for sensing a wide variety of parameters, including pressure, acceleration, and temperature, that can be formed as scalable arrays. As disclosed by these prior patent documents, whose contents are incorporated herein by reference, arrays of electromechanical switches are operable to close a contact if an input parameter exceeds a designed threshold to produce a digital output signal that results from current flowing through the closed contacts from an energy storage device. This mode of operation provides an ultra-low power scheme that is capable of using as little as about 10⁻¹² joules (pJ's) of energy from an energy storage device to produce a digital output signal for each event that results in the operation of a switch. The overall energy dissipation for an array of several thousand sensing elements (switches) is on the order of about 10⁻⁶ joules (μJ's), which is one hundred to one thousand times lower than state-of-the-art analog pressure or acceleration sensors coupled to analog-to-digital (ADC) circuits. Consequently, the power requirements of the modules 10 and 30 can be drastically reduced with the use of electromechanical switches, which in some cases can allow for the elimination of the need for a battery as the energy storage device, and allow for the use of capacitors and other relatively simple devices capable of storing energy. The use of electromechanical switches as the sensing elements 13 and 33 also enables the modules 10 and 30 to be operated to be fully event-triggered with standby current draws of less than 0.1 μA, and with fast wake-up and event capture response times on the order of a few micro-seconds.

In view of the above, a preferred aspect of the invention is that the arrays 12 and 32 of sensing elements 13 and 33 operate by extracting mechanical or thermal energy from the body in which the module 10 or 30 is implanted to close a contact, thereby closing a previously open electrical path, and produce a digital output signal through the resulting closed electrical path. This mode of sensing is capable of achieving considerable energy efficiency compared to conventional analog pressure, acceleration and temperature sensors. Another preferred aspect of the invention is that individual sensing elements 13 and 33 have different levels of sensitivity to the external input. For examples, the sensing elements 13 and 33 can be fabricated as switches that close at different threshold levels, such that different individual switches produce digital output signals in response to different input levels of the external input, with the result that the data stored in the memory 16 can be readily correlated to the overall level (amplitude) of the external input. The very small size to which the switches can be fabricated permits the integration of thousands of sensing elements 13 and 33 on a single chip measuring a few millimeters on a side using current NEMS and MEMS manufacturing processes, such that a wide range of amplitudes can be sensed.

The preferred operation for electromechanical switches for use as the sensing elements 13 and 33 in the present invention is to allow each individual switch to freely return to original open position after the level of the external input has dropped below the threshold level for the switch. In this manner, the duration that each switch is closed also provides a direct indication of the duration that the external input was above the threshold. As such, the controller 14 is able to process the outputs of all of the switches (elements 13 or 33) to not only generate data corresponding to the amplitude of an external input (for example, an impact), but also data corresponding to the duration of the external input. The controller 14 may also be operable to combine or integrate the amplitude and duration data according to a mathematical model, thereby reducing the amount of data that must be stored in the memory 16 and transmitted to a reader unit outside the module 10 or 30.

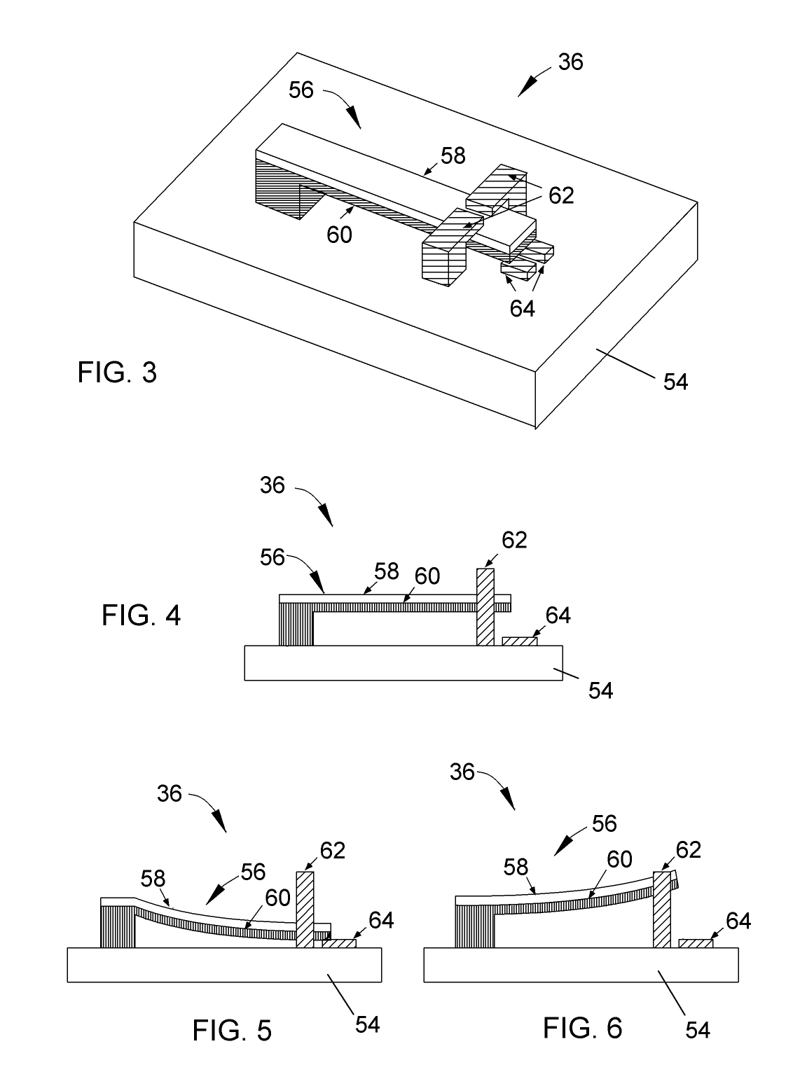

Exemplary but nonlimiting examples of MEMS and NEMS electromechanical switches capable of use with the present invention are represented in FIGS. 3 through 21. As discussed in more detail below, the switches include a moving microstructure that, by closing an electrical contact, creates a closed electrical path for producing an output that can be detected and processed by the controller 14 and stored in the memory 16. As noted previously, this operation is in response to an external input, such as a physiological or environmental parameter. Each switch defines an open electrical path and effectively has a threshold above which it closes a contact to create a closed electrical path.

In the embodiments of FIGS. 3 through 21, electromechanical switches 36 are represented as having various types of mechanical structures that move in response to an external environmental parameter such as vibrations, tilt, shock/acceleration, pressure, chemical levels, temperature, etc. This motion causes the mechanical structure, initially separated from one or more contacts to form an open electrical path, to contact one or more contacts to form a closed electrical path. Either the contacts or the mechanical structure may be connected to the energy storage device of the modules 10 and 30.

The movable mechanical structures of FIGS. 3 through 14 and 17 through 19 are cantilevered beams 56 fabricated directly on an integrated circuit substrate (e.g., CMOS wafer) 54 in which electronic devices (not shown) of the modules 10 and 30 can also be formed. An alternative is to fabricate the beams 56 on a separate substrate that is subsequently electrically coupled or bonded to the integrated circuit substrate 54. The beam 56 of the switch 36 shown in FIGS. 3 through 6 is configured as a temperature sensing element fabricated to include two thin films 58 and 60 having different coefficients of thermal expansion (CTE). Metals such as aluminum and gold are believed to be suitable for the films 58 and 60, respectively, though it is foreseeable that other material combinations could be used, including other metals and nonmetals. While the films 58 and 60 are shown as being positioned one atop the other to yield a vertical bimorph stack (vertical being normal to the surface of the substrate 54), it should be understood that the films 58 and 60 could be arranged side-by-side to yield a horizontal (lateral) bimorph stack (again, relative to the surface of the substrate 54). Furthermore, as understood by those skilled in the art, the beam 56 could include additional layers/films, such as adhesion layers to promote adhesion of the films 58 and 60 to each other, and stress compensation layers to improve the distribution of any processing-induced strain within the beam 56. As examples, if the films 58 and 60 are aluminum and gold, suitable adhesion layer materials include titanium and chromium, which in some cases may also be suitable for use as a stress compensation layer. It is also within the scope of the invention to pattern some of the layers that form the beam 56 for the purpose of modifying their properties, including response to temperature and/or other environmental conditions, electrical conductivity for use as electrical contacts, etc. As such, it should be understood that the beam 56 comprises layers of various materials that, in combination, yield a bimorphic effect One end of the beam 56 is anchored to the substrate 54, while the opposite end of the beam 56 is suspended between two sets of open contact pairs 62 and 64. The beam 56 may have electrically-conductive layers (not shown) for making electrical contact with the contact pairs 62 and 64. It can be readily appreciated that the structure of the switch 36 is simple and compatible with post-CMOS processing, and that very large, high-density arrays of such switches 36 can be fabricated in a very small area.

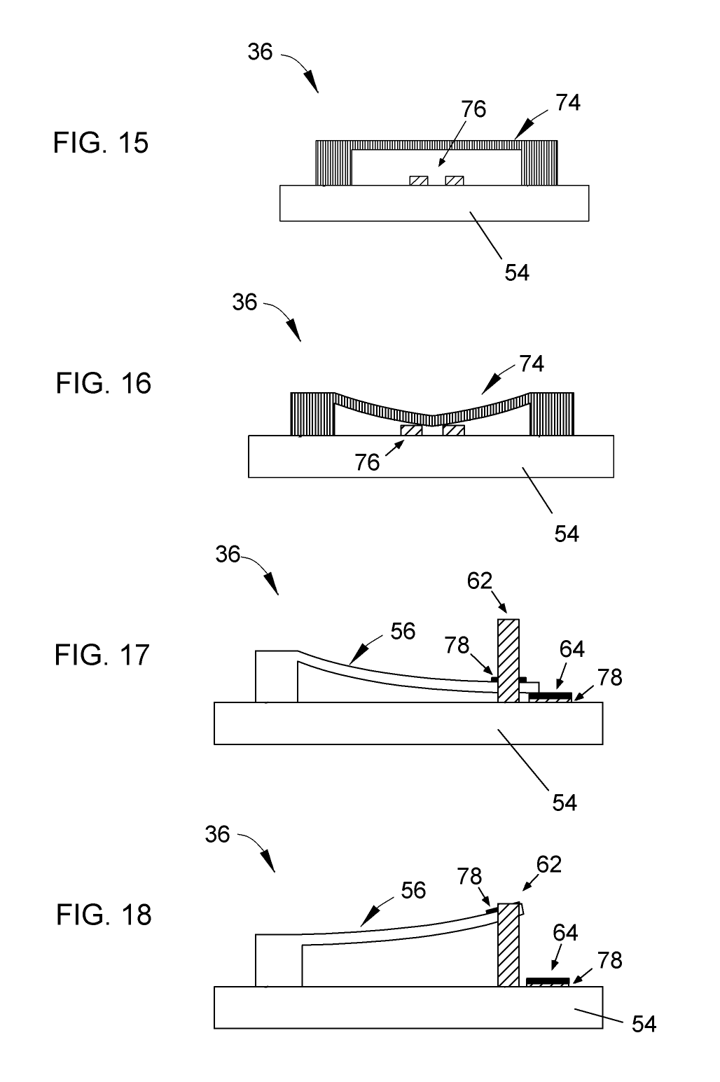

As a result of its multilayer bimorphic construction, the cantilevered beam 56 freely deflects with temperature change due to the CTE mismatch of the films 58 and 60. FIGS. 17 and 18 illustrate an example of the switch 36 of FIGS. 15 and 16 in which the beam 56 has a vertical bimorph stack, with its upper film 58 having a higher CTE than the lower film 60, for example, an aluminum film 58 over a gold film 60. A contact-mode switching function is provided when the portion of the beam 56 between the contact pairs 62 and 64 touches one of the pairs 62 or 64, completing an electrical path containing that particular pair 62 or 64. The temperature sensitivity of the cantilevered beam 56 of any given switch 36 can be analytically obtained based on structure geometries and material properties. Because sensitivity is independent of the beam width, the widths of the beams 56 of all switches 36 in a sensor array 12 or 32 of the modules 10 or 30 can be minimized to reduce the size of the array 12 or 32 to the extent that manufacturing reliability allows. FIGS. 5 and 6 represent the switch 36 at opposite extremes of its operating range corresponding to two threshold temperature conditions. In FIG. 17, the beam 56 has contacted and closed the lower contacts 64, whereas in FIG. 18 the beam 56 has contacted and closed the upper contacts 62. The direction of the beam deflection is determined by the input temperature being higher or lower than a predetermined temperature condition (i.e., room temperature, manufacturing temperature, etc.), and the difference between the CTE's of the films 58 and 60. In this manner, each switch 36 can be configured to have a switching function at a desired temperature setpoint (threshold). Furthermore, the sensor array 12 or 32 can contain switches 36 whose beams 56 are intentionally of different lengths, with longer beams 56 being more sensitive to temperature and resulting in contact with one of the sets of contact pairs 62 and 64 at progressively smaller temperature changes with increasing beam lengths. Scaling of the feature sizes of the beams 56 improves the achievable measurement resolution in addition to the die size reduction.

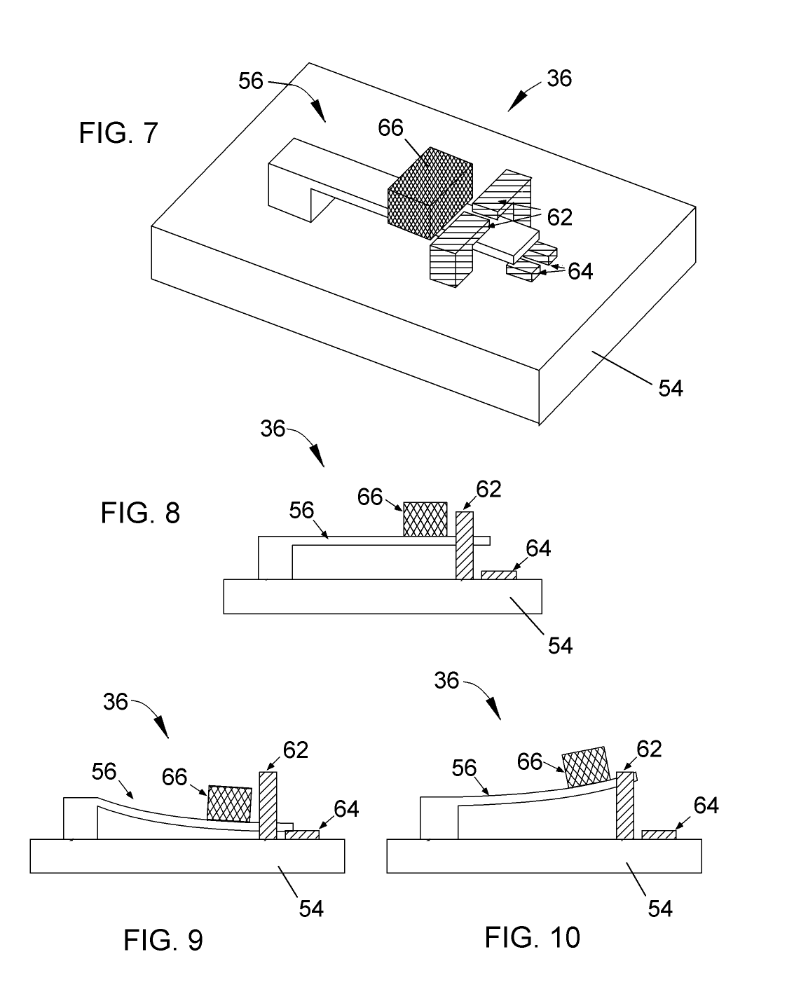

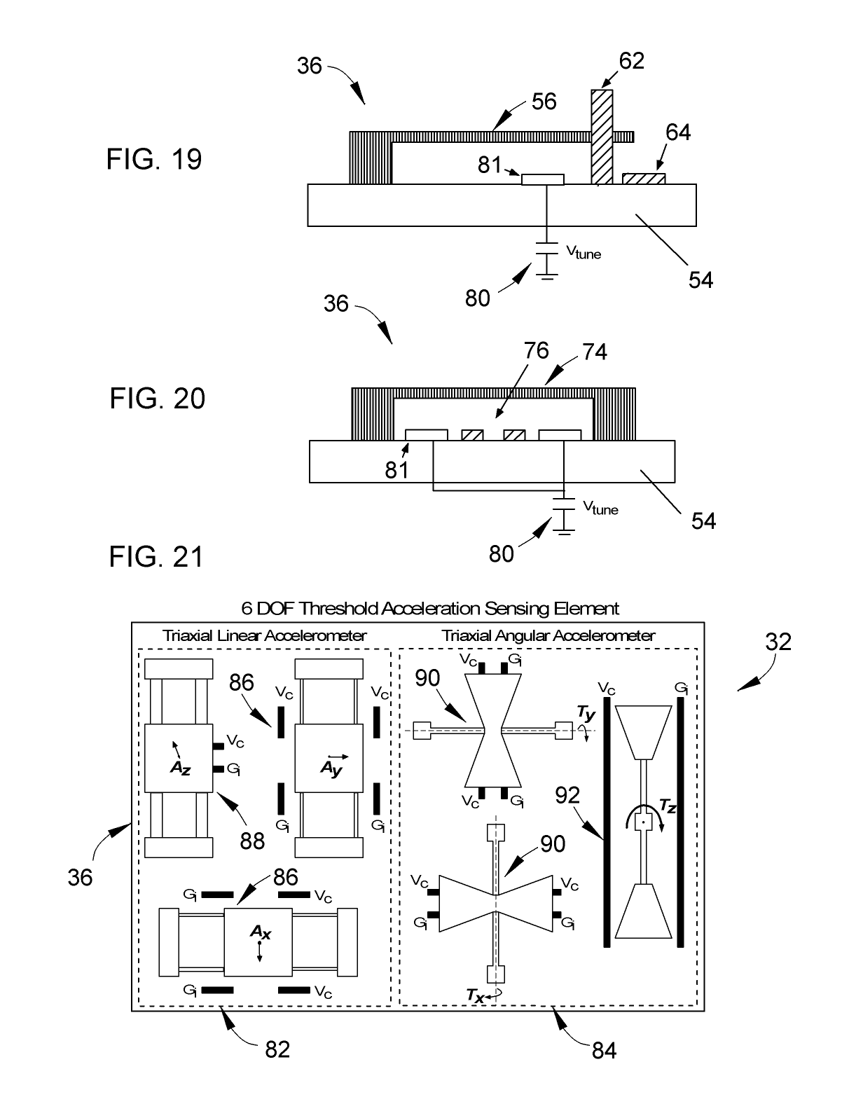

FIGS. 7 through 10 represent an impact/acceleration-sensing MEMS switch 36 that also operates using a cantilevered beam 56. As evident from FIGS. 19 through 22, the impact/acceleration-sensing switch 36 is similarly constructed to the temperature switch 36 of FIGS. 15 through 18, with the notable exception that the beam 56 is not required to be bimorphic or constructed of multiple materials. Instead, a proof mass 66 is mounted on the beam 56 to increase the responsiveness of the beam 56 to the impact and/or acceleration levels of interest. As with the temperature-sensing switch 36 of FIGS. 15 through 18, the impact/acceleration-sensing switch 36 of FIGS. 19 through 22 has two operating extremes that result in the beam 56 contacting either the upper or lower pair of contacts 62 and 64 in response to a threshold level of impact/acceleration, depending on the direction of the input impact or acceleration. For one skilled in the art, it is clear that other configurations and structures for impact/vibration switches can be used as well, an example of which is discussed below in reference to FIG. 21.

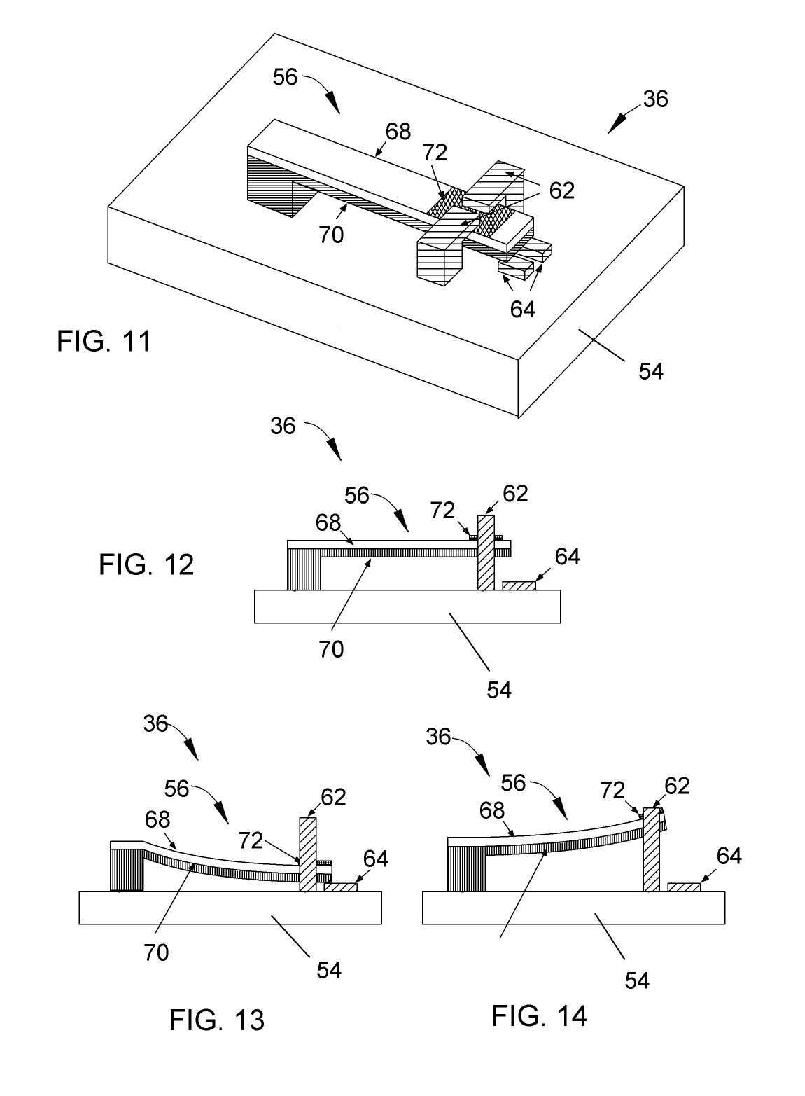

Also similar to the temperature switch 36 of FIGS. 15 through 18, FIGS. 11 through 14 represent a chemical-sensing MEMS switch 36 that operates on the basis of a bimorph effect using a cantilevered beam 56. In FIGS. 13 and 14, the beam 56 is shown at two operating extremes resulting in the beam 56 contacting either the upper or lower pair of contacts 62 and 64 in response to a threshold level of a chemical, depending on whether the sensed chemical concentration or level is higher or lower than the predetermined threshold. Deflection of the beam 56 and its threshold levels are dependent on two thin films 68 and 70 formed of materials that exhibit different chemical-induced expansion characteristics. For processing purposes, the lower film 70 can be formed of a thin metal film that does not exhibit any appreciable chemical-induced expansion. In contrast, the upper film 68 of the beam 56 is preferably formed of a material that exhibits a notable response to the chemical of interest. Because of the poor electrical conductance of certain materials that may be used to form the upper film 68, FIGS. 11 through 14 show the beam 56 is being provided with an electrically conductive layer 72 on that portion of the beam 56 that will contact the upper pair of contacts 62. As with the beam 56 of the temperature switch 36, the beam 56 of the chemical MEMS switch 36 can be formed to contain additional layers of a variety of different materials, both metallic and metallic, including adhesion-promoting, stress-distributing layers, and electrical contact layers, as well as patterned layers for the purpose of modifying the response of the beam 56 to chemical and other environmental conditions.

FIGS. 15 and 16 represent yet another embodiment for a switch 36, in which a diaphragm 74 is used in place of the cantilevered beams 56 discussed above. From FIGS. 15 and 16, it can be seen that the diaphragm 74 is supported above a pair of contacts 76, and that by forming the diaphragm 74, or at least its lower surface facing the contacts 76, of an electrically conductive material, a closed electrical path can be created across the contacts 76 if the ambient pressure above the diaphragm 74 meets or exceeds a threshold pressure. As well known in the art, the operation and sensitivity of the pressure-sensitive switch 36 of FIGS. 14 and 15 can be enhanced by evacuating the chamber formed by and between the diaphragm 74 and the substrate 54.

As previously noted, the beams 56 and diaphragm 74 can be configured to deflect while subjected to the external input, thereby producing a digital output that is detected and processed by the controller 14 and stored in the memory 16, and then return to their non-deflected positions once the external input is absent. Alternatively, the beams 56 and diaphragm 74 or their respective contacts 62, 64, and 76 may be connected to the energy management unit 18 so as to be maintained at different electrical voltages. As a result, once contact is made, the voltage difference can result in a sufficiently large electrostatic force that keeps the beam 56 or 74 in a closed position with its contacts. As represented with the beam 56 in FIGS. 17 and 18, by providing the beam 56 and/or its contacts 62 and 64 with thin dielectric layers 78, this voltage difference can be sufficiently high and sustained to keep the beam 56 pinned to the contacts 62 or 64 even after the sensed input parameter drops below the threshold for that parameter. As with the memory 16, the switches 36 can be provided with a reset capability by discharging the contact electrostatic capacitance that holds the mechanical structures to their contacts.

FIGS. 19 and 20 represent an approach for refining or calibrating the responses of the beams 56 and diaphragms 74 of the foregoing switches 36. In particular, FIGS. 19 and 20 depict a technique by which an adjustable electrical charge can be applied with an isolated capacitor 80 to one or more electrodes 81 placed in proximity to the beam 56 and diaphragm 74, enabling an adjustable electrostatic force to be applied that can bias (e.g., attract or repel) the beam 56 and diaphragm 74. In this manner, the deflection of the beam 56 and diaphragm 74 can be tuned so that contact with their corresponding contacts 62, 64, and 76 can be promoted or restrained.

FIG. 21 represents an acceleration sensor array 32 for use with the module 30, in which multiple different MEMS switches 36 provide a six-degree of freedom (DOF) acceleration sensing capability, with each switch 36 being capable of functioning similarly to that described for the impact/vibration switch 36 of FIGS. 7 through 10. In particular, one set of the switches 36 constitute a triaxial linear accelerometer array 82 that includes two lateral switches 86 and one out-of-plane switch 88, and a second set of switches 36 constitute a triaxial angular accelerometer array 84 that includes two torsional switches 90 having in-plane axes and a torsional switch 92 having an out-of-plane axis implemented by two in-plane linear proof masses with cantilever supports placed on opposite sides of a single common anchor. Contacts are placed along opposite sides of the torsional switch 92 such that a connection can be only made if the proof masses move in opposite directions to each other. As such, a linear acceleration has no effect on the torsional switch 92 because it moves both proof masses in the same direction and opposite contacts cannot be made.

By appropriately selecting the suspension beam, proof mass, and gap between the contacts, desired switching thresholds can be obtained for the switches 36 represented in FIG. 21. Cross-axis sensitivity can be minimized by proper suspension design and proof mass design. For instance, the angular torsional switches 90 and 92 may have pie-shaped proof masses to maximize their response to external torque and minimize their linear response. The out-of-plane linear switch 88 requires a top contact (not shown) for bidirectional operation, which can be formed on a structure that also serves as an out-of-plane impact stop for all of the switches 86, 88, 90, and 92 in all axes.

In view of the foregoing, it should be appreciated that the modules 10 and 30 can be produced using post-CMOS mass production MEMS technologies. To further reduce package size and external parasitic impedance, the micro- or nano-electromechanical (MEMS or NEMS) sensing elements 13 and 33 can be integrated directly on the integrated circuit chip on which the circuitry for the controller 14 is fabricated. The sensing elements 13 and 33 can be fabricated subsequent to forming the CMOS integrated circuits, and then integrated onto the surface of the CMOS chip, or fabricated and attached to the CMOS chip using techniques such as flip-chip bonding, wire-bonding or other methodologies known to those skilled in the art. Encapsulation of the circuits and sensing elements 13 and 33 may be achieved using any of a variety of techniques, but is not limited to solder bonding, gold eutectic bonding, fusion bonding, polymer bonding, or any other technique known to those skilled in the art. Wafer-level packaging of the modules 10 and 30 can be employed to reduce costs and seal the components of the modules 10 and 30 from the damaging effects of the body. By hermetically sealing the packaging, the modules 10 and 30 can be implanted for many years.

Power efficient digital signal processing enabled by the digital outputs of an array of switches can be employed to provide flexibility and programmability, in conjunction with extended features such as on-chip calculations capable of correlating the injury to the recorded parameters. While many sensing systems and research utilize peak impact to determine levels of head trauma, it has been determined that both amplitude of impact and duration are important for determining the level of head trauma. Models such as Head Injury Criterion (HIC), which is currently used to evaluate the efficacy of helmets, provide output based upon mathematical models that factor in both levels of impact and duration criteria. As previously discussed, the implantable modules 10 and 30 of this invention can have the capability of recording both amplitudes and durations of impacts, and the controller 14 can be used to combine and integrate amplitude and duration data based on the mathematical model employed to calculate HIC. As such, the data retrieved from the modules 10 and 30 can be directly employed to predict the likelihood or risk of injury resulting from one or more impacts suffered by an individual.

The modules 10 and 30 are also well suited for use in head trauma monitoring systems. Such a capability is of particular interest in view of investigations concerning the long term effects of multiple mild traumatic brain injuries (TBIs). Postmortem studies of the brains of American football players that have suffered from multiple concussions have shown that there is widespread damage throughout the brain. The brain tissue damage in autopsied brains appeared similar to tissue from patients suffering from Alzheimer's disease, even though many of the subjects were otherwise young and healthy. In these subjects, none of this damage appeared on MRI or CT scans, yet damage due to concussions can affect parts of the brain that effect emotion, rage, etc. and it has been found that the even mild TBI can kill brain cells and neural connections. Occurrence of mental disorders including major depression and attention deficit in people that have suffered from multiple concussions has been shown to be common. It is anticipated that early detection and treatment of head impacts would provide improved recovery from these injuries. For example, recent studies have shown that certain Alzheimer's disease medications can be helpful in reducing the damage caused by TBI if treated in a timely manner. Certain embodiments of the modules 10 and 30 of this invention are capable of monitoring head trauma to quickly and accurately determine level of trauma, which enables medical personnel to more accurately assessment of injury, improving treatment methodologies through early intervention.

From the foregoing, it will be appreciated that modules 10 and 30 with the low power dissipation capabilities described above can be adapted for use in a wide variety of applications that can be implemented with wired and wireless sensor modules, or used in conjunction with passive and active RFID tags for RFID-based sensors. Therefore, while the invention has been described in terms of particular embodiments, it is apparent that other forms could be adopted by one skilled in the art. For example, the physical configurations and uses of the modules 10, switches 36, etc., could differ from that shown and described, and materials and processes other than those noted could be use. Therefore, the scope of the invention is to be limited only by the following claims.

1. A method of monitoring impacts to which a living body is subjected, the method comprising:
providing an implantable sensing module that comprises a rigid housing containing at least one energy storage device, at least one electromechanical sensing element that is responsive to impacts, means for generating outputs corresponding to impacts to which the electromechanical sensing element is subjected, and means for recording data corresponding to the outputs;
implanting the module in a living body so that the module is located internally within the living body and is connected to a rigid portion of the living body chosen from the group consisting of bones and teeth;
monitoring impacts to the living body by monitoring a level of at least one impact to which the electromechanical sensing element is subjected within the living body;
producing an output corresponding to the level of the at least one impact sensed by the electromechanical sensing element;
storing data in the recording means within the module corresponding to the output of the electromechanical sensing element; and then
wirelessly retrieving the data stored in the recording means while the module remains implanted in the living body.

2. The method according to claim 1, wherein the recording means records the data without being supplied power external of the module.

3. The method according to claim 1, wherein the recording means comprises nonvolatile digital memory devices.

4. The method according to claim 1, wherein the energy storage device comprises a battery.

5. The method according to claim 1, wherein the energy storage device comprises an electrical capacitive element.

6. The method according to claim 1, wherein the at least one electromechanical sensing element comprises an accelerometer.

7. The method according to claim 1, wherein the at least one electromechanical sensing element comprises a plurality of integrated electromechanical switches, the electromechanical switches define open electrical paths when not subjected to an impact and are operable to define closed electrical paths that produce the outputs, the electromechanical switches define the closed electrical paths in response to impacts and produce the outputs while the electromechanical switches are subject to impacts that exceed thresholds of the electromechanical switches, and the electromechanical switches define the open electrical paths and do not produce the outputs when no longer subject to impacts that exceed the thresholds of the electromechanical switches.

8. The method according to claim 7, wherein the electromechanical switches have different thresholds so as to have different levels of sensitivity to impacts.

9. The method according to claim 8, wherein the outputs produced by the electromechanical switches are used to calculate an amplitude of an impact determined from the different thresholds of the electromechanical switches.

10. The method according to claim 8, wherein the outputs produced by the electromechanical switches are used to determine the duration of an impact.

11. The method according to claim 8, wherein the data stored in the recording means comprises both duration and amplitude of an impact.

12. The method according to claim 11, wherein the data are calculated using a mathematical function implemented electronically within the module.

13. The method according to claim 11, further comprising predicting injury to the living body based on the data retrieved from the module.

14. The method according to claim 1, wherein the housing of the module further comprises at least one additional sensing element chosen from the group consisting of pressure and temperature sensing elements.

15. The method according to claim 14, wherein the at least one additional sensing element comprises a plurality of integrated electromechanical switches, the electromechanical switches define open electrical paths and are operable to define closed electrical paths in response to pressure or temperature.

16. The method according to claim 15, wherein the electromechanical switches have different thresholds at which the electromechanical switches define the closed electrical paths so as to have different levels of sensitivity to pressure or temperature.

17. The method according to claim 16, further comprising recording the duration over which each of the electromechanical switches defines the closed electrical path thereof in response to pressure or temperature.

18. The method according to claim 1, wherein the module is implanted by attaching the module to a bone of the living body.

19. The method according to claim 18, wherein the bone is the skull of the living body.

20. The method according to claim 1, wherein the module is implanted by attaching the module to a tooth of the living body.

21. A method of monitoring at least one external input chosen from the group consisting of physical parameters of a living body and environmental parameters to which the living body is subjected, the method comprising:
providing an electromechanical module that comprises at least one integrated energy storage device and a plurality of integrated electromechanical switches, the electromechanical switches defining open electrical paths and being operable to define closed electrical paths to produce outputs in response to the external input, and the electromechanical switches having different levels of sensitivity to the external input;
implanting the module in a living body;
subjecting the living body to the external input that causes at least two of the electromechanical switches to define at least two of the closed electrical paths in response to different input levels of the external input, the at least two closed electrical paths producing at least two outputs corresponding to the different input levels of the external input; and
obtaining data from the module corresponding to the outputs of the electromechanical switches.

22. The method according to claim 21, wherein the electromechanical switches comprise movable microstructures capable of physical movement between open positions that define the open electrical paths and closed positions that define the closed electrical paths, and the movable microstructures move from the open positions to the closed positions in response to the different input levels of the external input.

23. The method according to claim 22, wherein the movable microstructures comprise cantilevered beams and the cantilevered beams deflect from the open positions to the closed positions in response to the different input levels of the external input.

24. The method according to claim 22, wherein the movable microstructures comprise diaphragms and the diaphragms deflect from the open positions to the closed positions in response to the different input levels of the external input.

25. The method according to claim 20, further comprising storing the data in memory devices.

26. The method according to claim 25, wherein the data comprise the amplitude of the external input.

27. The method according to claim 25, wherein the data comprise the amplitude and duration of the external input.

28. The method according to claim 27, wherein the outputs of the electromechanical switches comprise a combination of amplitude and duration of the external input, and the data are calculated using a mathematical function implemented electronically within the module.

29. The method according to claim 25, wherein the memory devices comprise nonvolatile digital memory devices.

30. The method according to claim 25, further comprising wirelessly retrieving the data stored in the memory devices while the module remains implanted in the living body.

31. The method according to claim 25, further comprising discharging the memory devices after the data are wirelessly retrieved therefrom.

32. The method according to claim 25, wherein the memory devices store the data without being supplied power external of the module.

33. The method according to claim 21, further comprising wirelessly charging the energy storage device while the module remains implanted in the living body.

34. The method according to claim 21, wherein the external input is at least one physical or environmental parameter chosen from the group consisting of temperature, relative humidity, chemicals, motion, impact, vibration, orientation, pressure, acceleration, and biological agents.

35. An implantable sensing module for monitoring impacts to which a living body is subjected, the sensing module comprising:
a housing;
at least one energy storage device within the housing;
at least one set of electromechanical sensing elements within the housing, the electromechanical sensing elements being responsive to impacts, each of the electromechanical sensing elements defining an open electrical path when not subjected to an impact and operable to define a closed electrical path that produces an output in response to an impact only while the electromechanical sensing element is subject to the impact and if the impact exceeds a threshold of the electromechanical sensing element, and then again defining the open electrical path thereof so as not to produce an output when no longer subject to the impact that exceeded the threshold thereof;
means within the housing for generating data corresponding to the outputs of the electromechanical sensing elements; and
means within the housing for recording the data;
wherein the electromechanical sensing elements, the generating means, and the recording means are powered only by the energy storage device when, respectively, producing the output, generating the data, and recording the data in response to an impact that exceeded the threshold of one or more of the electromechanical sensing elements.

36. The implantable sensing module according to claim 35, wherein the sensing module is implanted in a living body so that the housing is connected to a rigid portion of the living body chosen from the group consisting of bones and teeth.

37. The implantable sensing module according to claim 35, further comprising means for wirelessly retrieving the data stored in the recording means while the sensing module remains implanted in the living body.

38. The implantable sensing module according to claim 35, wherein the recording means comprises nonvolatile digital memory devices.

39. The implantable sensing module according to claim 35, wherein the set of electromechanical sensing elements comprises a plurality of integrated electromechanical switches, the electromechanical switches are open to define the open electrical paths when not subjected to an impact and close to define the closed electrical paths that produce the outputs while subject to impacts that exceed the thresholds thereof.

40. The implantable sensing module according to claim 35, wherein the electromechanical sensing elements have different thresholds so as to have different levels of sensitivity to impacts.

41. The implantable sensing module according to claim 35, wherein the data stored in the recording means comprises both duration and amplitude of an impact.

42. The implantable sensing module according to claim 41, further comprising means within the housing for processing the amplitude and duration data and predicting the likelihood or risk of injury resulting from impacts.
